(19) United States
(12) Patent Application Publication
(10) Pub. No.: US 2018/0250314 A1
(43) Pub. Date: Sep. 6, 2018

(54) Skin Treatment System

(71) Applicant: Lori HADDAD, Troy, MI (US)

(72) Inventor: Lori HADDAD, Troy, MI (US)

(21) Appl. No.: 15/907,580

(22) Filed: Feb. 28, 2018

Related U.S. Application Data
(60) Provisional application No. 62/465,311, filed on Mar. 1, 2017.

(51) Int. Cl. A61K 31/60; A61K 31/19; A61P 17/10; A61P 17/00; A61K 9/00

(52) U.S. Cl. CPC A61K 31/60; A61K 31/19; A61K 9/0014; A61P 17/00; A61P 17/10

(57) ABSTRACT

A system for skin treatment including first, second, third, fourth, and fifth formulations that each include glycolic acid in an amount that ranges between 6 to 13 wt %, and salicylic acid in an amount that ranges between 1.3 to 2 wt %, wherein each of the first, second, third, fourth, and fifth formulations are free of ethanolamine, paraben, propylene glycol, and sulfate.

### CROSS-REFERENCE TO RELATED APPLICATIONS

This application claims the benefit of U.S. Provisional Application No. 62/465,311, filed on Mar. 1, 2017. The entire disclosure of the above application is incorporated herein by reference.

### FIELD

The present disclosure relates to a plurality of topical treatment formulations for treating various skin conditions.

### BACKGROUND

This section provides background information related to the present disclosure which is not necessarily prior art.

Treating skin conditions is a multi-billion dollar industry that provides skin-treatment products such as prescription medications, and over the counter (OTC) cosmetics, creams, and lotions. Patients, however, have become apprehensive of these medications and treatments due to their often detrimental side effect profile, their inclusion of harsh chemical ingredients, and extreme reactivity to sensitive skin.

One example skin condition that patients actively seek solutions for is acne vulgaris (acne), which refers to inflammation of hair follicles and sebaceous glands. Acne is the most common cutaneous disorder in adolescents, but can be seen in any age group. Follicular rupture, which releases pro-inflammatory lipids and keratin into the dermis, can intensify the inflammation. Acne occurs on areas of the body with hormonally sensitive sebaceous glands, such as the face, neck, chest, and back. It usually decreases in severity with age, but certain conditions like pregnancy, immunosuppression, stress and hormonal imbalances can cause exacerbations of acne.

Common treatment products that are used to treat acne can be OTC (over-the-counter) medications such as lotions or creams that include Benzyl Peroxide (BP), or prescription strength medications that also include BP along with the addition of antibiotics (e.g., benza-clin, benza-mycin, and the like). These treatments, however, contain drawbacks because unlike natural products, these products have various side effects and safety risks. For example, BP is a pregnancy category C compound that is known to cause hypo-pigmentation, which renders it unsafe for all ethnicities. Another widely used acne product is tretinoin, also known as Retin-A®. This vitamin A derivative is a strong irritant to sensitive skin, and is also a pregnancy category C compound such that most dermatologists do not recommend its use in pregnant women due to its safety risks on the fetus.

Moreover, most OTC and topical skin products contain numerous synthetic ingredients that consist of skin irritants, endocrine disrupters, skin penetrators, and have a carcinogenic profile. Example synthetic ingredients include parabens, synthetic colors, fragrances, phthalates, sodium lauryl sulfate, formaldehyde, propylene glycols, and sunscreen chemicals (e.g., benzophenone, PABA, avobenzone, homosalate and ethoxycinnmate), to name a few. Hence the need for a skin treatment system that is natural, inexpensive, and eliminates the dangerous side effects commonly encountered with known topical medications that include BP is essential.

### SUMMARY

This section provides a general summary of the disclosure, and is not a comprehensive disclosure of its full scope or all of its features.

The present disclosure provides a skin treatment system including a plurality of different formulations for use at different times throughout the day, which is superior and safer in controlling acne when compared to several OTC and prescription strength medications. The “safety” of the formulation is superior, and the formulations are certified as “green” products that are natural and free of synthetic additives. Each formulation utilizes natural products that are relatively inexpensive and gentle, yet effective on all skin types including sensitive skin, with a superior safety profile for all ethnicities, all ages, and gestational term. Each formulation, in particular, utilizes an all-natural glycolic acid and salicylic acid combination, in addition to other natural ingredients. Both glycolic acid and salicylic acid have been shown to benefit acne treatment when used alone, topically. When these compounds are used in combination, the efficacy on common skin conditions like acne vulgaris, keratosis pilaris, melasma, post-inflammatory hyper-pigmentation, rosacea, seborrhic dermatitis, and folliculitis is improved.

Further areas of applicability will become apparent from the description provided herein. The description and specific examples in this summary are intended for purposes of illustration only and are not intended to limit the scope of the present disclosure.

### DETAILED DESCRIPTION

As noted above, the skin treatment system includes a plurality of different formulations that are useful, as well as safer, for treating common skin conditions like acne vulgaris, keratosis pilaris, melasma, post-inflammatory hyper-pigmentation, rosacea, seborrhic dermatitis, and folliculitis. As also noted above, acne vulgaris is an inflammation of hair follicles and sebaceous glands.

Keratosis Pilaris (KP) is a common disorder of follicular keratinization that is characterized by keratotic follicular papules and perifollicular erythema. KP typically occurs during childhood or adolescence with spiny papules predominantly involving the extensors of arms and the thigh. It is important to note, however, that any area may be affected, and it can be seen in association with other skin conditions like Atopic Dermatitis.

Melasma is an acquired hyper-pigmentation that occurs on sun-exposed areas, and can be cosmetically debilitating when it occurs on the face. Melasma is usually seen during pregnancy, but can also be seen in women using oral contraceptives, individuals with darker skin tones, and sometimes in men. Although melasma cannot be cured, it can be controlled.

Post-inflammatory hyper-pigmentation is characterized by the development of hyper-pigmented macules or patches in the distribution of a preceding inflammatory dermatosis, such as acne. Patients having this condition have a propensity to “pick” on the pustule, which can lead to scar formation.

Rosacea is an inflammatory facial skin disorder characterized by erythematous papules and pustules, without comedones. It is associated with an unpredictable facial redness, and oftentimes occurs without an identifiable trigger. Known triggers, however, include the use of alcohol or medication, and stress. Rosacea is also the leading cause of acne in adults.

Folliculitis is a bacterial infection of the hair follicles, which can cause follicular plugging, itching, and pustule formation. The condition may occur anywhere on the skin, with the exception of the palms and soles.

The skin treatment system according to the present disclosure includes various formulations that each includes glycolic acid and salicylic acid. Glycolic acid and salicylic acid are each pregnancy category B compounds, with minimal absorption through the dermis, and a moderate lactation rating and, therefore, are considered safe for use during pregnancy and breastfeeding, respectively. Further, these compounds may be used by patients of all ethnicities and skin tones because use of these compounds in the formulations does not appear to cause hypo- or hyper-pigmentation. Thus, children, adults, pregnant women, lactating women, African-Americans, and all ethnic groups benefit from the use of these natural and green products that are safe and effective for each individual, while indifferent to age, race, gestational and breastfeeding term.

Glycolic acid is a natural compound that may be extracted from sugar cane, and is part of a set of molecules known as Alpha Hydroxy Acids (AHA). There are five types of AHAs including glycolic acid (produced from, for example, sugar cane), lactic acid (produced from, for example, milk), citric acid (produced from, for example, oranges and lemons), malic acid (produced from, for example, apples and pears), and tartaric acids (produced from, for example, grapes).

During use of the skin treatment formulations, the glycolic acid is configured to react with the top layer of skin, i.e., the stratum corneum, by breaking it down while dissolving sebum and other substances that bind dead tissue together. The dead skin cells produced when exposed to the glycolic acid may then be sloughed off with gentle exfoliation, which clears blocked pores and opens comedones to reveal smoother, brighter, younger-looking skin. This property of the glycolic acid has been demonstrated in our in-vivo study, where its use on patients with keratosis pilaris revealed clearer and smoother skin on arms, buttocks, thighs, and cheeks. The Glycolic Acid exfoliates the stratum corneum, thereby unplugging the hair follicle that is clogged by dead tissue, revealing smoother, clearer skin devoid of keratin clogged hair follicule that can give the “goose bump” appearance on skin.

Glycolic acid has also been shown to reduce fine lines and signs of premature aging by increasing cell turnover. In addition, scars also appear to respond well to consistent treatment with glycolic acid.

Salicylic acid is also a natural compound, and acts as an exfoliant. Salicylic acid is formed from a complex carbohydrate found in willow bark. It is effective at low concentrations, and is significantly less irritating than other products like benzyl peroxide and retinoids. A mild solution of salicylic acid applied directly to the skin results in similar benefits of scrubbing, without the risk of rupturing pores or disrupting subcutaneous vasculature. Studies have pointed to its skin brightening properties, without the use of harmful bleaching agents like hydroquinone.

Melasma, a sun-damaged hyper-pigmentation of skin, has no cure; however control of the hyper-pigmentation has been successfully obtained in our in-vivo study using the “combo” treatment of glycolic acid with salicylic acid. Patients noticed a combined brightening, lightening, and tightening effect on their treated skin.

Seborrheic Dermatitis affects all ages including infants (cradle cap), adolescents (affecting face, scalp, chest, back, and genitals), and adults. Patients in our in-vivo study noticed significant improvement of their skin condition with decreased scaling, decreased erythema, and decreased pruritus, hence a clear skin without the side effects of topical steroid usage. Rosacea patients and Acne Vulgaris patients had noticeable improvements of their dermis with decreased erythema, decreased comedones and cysts, and effective control of flaring term. On a side note, when used alone, salicylic acid is most effective when applied continuously even after the acne has cleared.

Each of the formulations according to the present disclosure is provided below. As noted above, the various formulations can be used at different times throughout the day. For example, formulations including an increased amount of glycolic acid and salicylic acid can be used in the morning and evening, while formulations including lesser amounts of these materials can be used throughout the day, or vice versa. Alternatively, these formulations can be used in a staggered fashion where formulations having increased amounts (or decreased amounts) of these materials are used first, and then formulations having decreased amounts (or increased amounts) of these materials are used second, and so on throughout the day. Alternatively, a single formulation may be used only a single time during the day, or used multiple times throughout the day. Regardless, each patient can be given a specialized treatment protocol, whether it includes a single formulation or multiple formulations that is specifically adapted for the particular patient.

Formulation 1: PURIFIED WATER, GLYCOLIC ACID (6%), GLYCERIN, PYRIDOXINE HYDROCHLORIDE (VITAMIN B6), SALYCILIC ACID (1.3%), HYDROLYZED COLLAGEN, SODIUM ASCORBYL PHOSPHATE (VITAMIN C), ALLANTOIN, ALOE BARBADENSIS (ALOE VERA) LEAF EXTRACT, CELLULOSE GUM, HYDROLYZED WHEAT PROTEIN, CITRIC ACID, ISOPROPYL ALCOHOL, and SODIUM HYDROXIDE (TO ADJUST pH).

Formulation 2: PURIFIED WATER, GLYCOLIC ACID (6%), GLYCERIN, PYRIDOXINE HYDROCHLORIDE (VITAMIN B6), SALYCILIC ACID (2%), HYDROLYZED COLLAGEN, SODIUM ASCORBYL PHOSPHATE (VITAMIN C), ALLANTOIN, ALOE BARBADENSIS (ALOE VERA) LEAF EXTRACT, CELLULOSE GUM, HYDROLYZED WHEAT PROTEIN, CITRIC ACID, ISOPROPYL ALCOHOL, SODIUM HYDROXIDE (TO ADJUST pH).

Formulation 3: PURIFIED WATER, EUGENIA CARYOPHYLLUS (CLOVE) FLOWER EXTRACT, GLYCOLIC ACID (8%), GLYCERIN, ISOPROPYL ALCOHOL, CUCUMIS SATIVUS (CUCUMBER) FRUIT EXTRACT, SALICYLIC ACID (1.3%), PROVITAMIN B5 (D-PANTHENOL), CELLULOSE GUM, HYDROLYZED WHEAT PROTEIN, CITRIC ACID, SODIUM HYDROXIDE (TO ADJUST pH).

Formulation 4: PURIFIED WATER, EUGENIA CARYOPHYLLUS (CLOVE) FLOWER EXTRACT, GLYCOLIC ACID (8%), GLYCERIN, ISOPROPYL ALCOHOL, CUCUMIS SATIVUS (CUCUMBER) FRUIT EXTRACT, SALICYLIC ACID (2%), PROVITAMIN B5 (D-PANTHENOL), CELLULOSE GUM, HYDROLYZED WHEAT PROTEIN, CITRIC ACID, SODIUM HYDROXIDE (TO ADJUST pH).

Formulation 5: PURIFIED WATER, GLYCOLIC ACID (13%), GLYCERIN, CUCUMIS SATIVUS (CUCUMBER) FRUIT EXTRACT, SALICYLIC ACID (1.3%), PROVITAMIN B5 (D-PANTHENOL), CELLULOSE GUM, HYDROLYZED WHEAT PROTEIN, PHENOXYETHANOL, CAPRYLYL GLYCOL, ALOE BARBADENSIS (ALOE VERA) LEAF EXTRACT, HYALURONIC ACID, ISOPROPYL ALCOHOL, SORBIC ACID, SODIUM HYDROXIDE (TO ADJUST pH).

As can be seen from the above, each formulation includes glycolic acid in an amount that ranges between 6% by weight to 13% by weight, and each formulation includes salicylic acid in an amount that ranges between 1.3% by weight to 2% by weight. It should be understood, however, that other ranges are contemplated by the present disclosure. In general, the amount of glycolic acid should range between 4% by weight to 15% by weight, and the amount of salicylic acid should range between 1% by weight to 3% by weight. In addition, each of the above-noted formulations are free of synthetic additives.

### Experimental Data

A retrospective study was conducted to assess the efficacy of the various formulations, which included patients with mild to moderate inflammatory and cystic acne, rosacea, folliculitis and KP that would be appropriate to treat with prescription strength medication and who were willing to stop using all other treatments. Excluded patients included those on chemotherapeutic agents for actinic keratosis, exhibited a sensitivity to salicylic or glycolic acid, or those who were unwilling to stop concomitant treatment. Sixty-six patients were selected ranging in age from 17 to 46. None of the candidates were lost through to follow up. The patients were asked to apply a selected formulation at night for two weeks. At the follow-up appointment, the patients were asked to fill out a questionnaire regarding the changes they have noticed with their skin.

### Results

Over 90% of the patients reported significant overall improvement in acne with decreases in comedonal and cystic acne. 70-80% of the patients reported a decrease in skin oiliness, an increase in even texture, and smoother, more radiant skin. Physical exam findings were consistent with patient findings. For instance, one patient stopped using the wake up serum (FORMULATION 1) after one week due to mild itchiness. Compliance was assessed based on previous patient records. No new patients were selected for the study.

In conclusion, the formulations according to the present application including glycolic acid and salicylic acid in amounts that range between 6-13% by weight and 1.3 to 2% by weight, respectively, work synergistically together to achieve quick results. The products showed superior improvement in skin conditions in comparison to leading acne prescriptions and over the counter preparations, and patient satisfaction was largely attributed to an improvement in skin texture, tone, a decrease in hyper-pigmentation, and oil production. In addition, the formulations are deemed safe in all ethnicities, all ages, all genders, and during pregnancy. The formula is all natural, and is allergen-free, dye-free, ethanolamine free, paraben free, propylene glycol free and sulfate free (chemical additives free). Thus, patients with mild to moderate inflammatory and cystic acne, melasma, oily skin types, keratosis pilaris, seborrhea dermatitis, pseudo-folliculitis barbae, folliculitis, and rosacea can achieve alleviation of these conditions using the formulations according to the present disclosure. Since it is mild on the skin with hydrating, cleansing and exfoliative properties, continuous use even after resolution of the primary condition is recommended.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for skin treatment, comprising a first formulation including glycolic acid in an amount that ranges between 6 to 13 wt %, and salicylic acid in an amount that ranges between 1.3 to 2 wt %,
wherein the first formulation is free of ethanolamine, paraben, propylene glycol, and sulfate.

2. The system for skin treatment according to claim 1, further comprising a second formulation including glycolic acid in an amount that ranges between 6 to 13 wt %, and salicylic acid in an amount that ranges between 1.3 to 2 wt %,
wherein the second formulation is free of ethanolamine, paraben, propylene glycol, and sulfate.

3. The system for skin treatment according to claim 2, further comprising a third formulation including glycolic acid in an amount that ranges between 6 to 13 wt %, and salicylic acid in an amount that ranges between 1.3 to 2 wt %,
wherein the third formulation is free of ethanolamine, paraben, propylene glycol, and sulfate.

4. The system for skin treatment according to claim 1, wherein, in the first formulation, the amount of glycolic acid is about 6 wt %, and the amount of salicylic acid is about 1.3 wt %.

5. The system for skin treatment according to claim 2, wherein, in the second formulation, the amount of glycolic acid is about 6 wt %, and the amount of salicylic acid is about 2.0 wt %.

6. The system for skin treatment according to claim 3, wherein, in the third formulation, the amount of glycolic acid is about 8 wt %, and the amount of salicylic acid is about 1.3 wt %.

7. The system for skin treatment according to claim 3, further comprising a fourth formulation including glycolic acid in an amount that ranges between 6 to 13 wt %, and salicylic acid in an amount that ranges between 1.3 to 2 wt %,
wherein the fourth formulation is free of ethanolamine, paraben, propylene glycol, and sulfate.

8. The system for skin treatment according to claim 7, wherein, in the fourth formulation, the amount of glycolic acid is about 8 wt %, and the amount of salicylic acid is about 2.0 wt %.

9. The system for skin treatment according to claim 7, further comprising a fifth formulation including glycolic acid in an amount that ranges between 6 to 13 wt %, and salicylic acid in an amount that ranges between 1.3 to 2 wt %,
wherein the fifth formulation is free of ethanolamine, paraben, propylene glycol, and sulfate.

10. The system for skin treatment according to claim 7, wherein, in the fourth formulation, the amount of glycolic acid is about 13 wt %, and the amount of salicylic acid is about 1.3 wt %.

11. The system for skin treatment according to claim 10, wherein each of the first, second, third, fourth, and fifth formulations are used to treat at least one condition selected from the group consisting of acne vulgaris, keratosis pilaris, melasma, post-inflammatory hyper-pigmentation, rosacea, seborrhic dermatitis, and folliculitis.
